(19) United States
(12) Patent Application Publication
(10) Pub. No.: US 2023/0301619 A1
(43) Pub. Date: Sep. 28, 2023

(54) Systems and Methods for Endovascularly Restoring Venous Valve Function

(71) Applicant: Bard Peripheral Vascular, Inc., Franklin Lakes, NJ (US)

(72) Inventors: Bryon Pelzek, Gold Canyon, AZ (US); Casey Rockwood, Phoenix, AZ (US); Himanshu Suri, Laveen, AZ (US); Joseph Sansone, Paradise Valley, AZ (US); Mandi Frost, Mesa, AZ (US); Matt Casiraro, Tempe, AZ (US); Sheena Moore, Scottsdale, AZ (US); Sherese L. Hill, Mesa, AZ (US)

(21) Appl. No.: 18/019,772

(22) PCT Filed: Aug. 6, 2020
(86) PCT No.: PCT/US2020/045247

§ 371 Date: Feb. 3, 2023

(51) Int. Cl. A61B 8/08; A61B 17/34; A61B 90/00

(52) U.S. Cl. CPC A61B 8/0833; A61B 17/3403; A61B 90/36; A61B 2090/378

(57) ABSTRACT

Systems and methods are disclosed for restoring venous valve function. In some embodiments, for example, a system includes an implantable coil and a deployment device therefor. The implantable coil has a non-coiled configuration in a first state and a coiled configuration in a second state configured to compress incompetent valves. The deployment device includes a handle, an outer sheath, a needle, and a mandrel. The handle includes a first actuator and a second actuator. The outer sheath includes a side opening proximate a distal end. The needle is operatively coupled to the first actuator. The mandrel is operatively coupled to the second actuator. The mandrel is configured to push the implantable coil through a needle tip of the needle, which allows the implantable coil to transition from the first state within the deployment device to the second state outside of the deployment device such as around a vein.

### BACKGROUND

Chronic venous insufficiency (“CVI”) is the most common disease in the world. CVI is estimated to affect about 40% of the population in the United States alone.

CVI is a consequence of deep-vein valvular incompetence in the legs. When a valve properly functions as shown in FIG. 7A, valve leaflets meet and close the valve preventing retrograde blood flow therethrough. However, when the valve leaflets do not meet as shown in FIG. 7B, the valve is incompetent and cannot properly function leaving the valve open to valvular reflux and retrograde blood flow therethrough. Improperly functioning valves can lead to swelling in the legs, as well as formation of varicose veins and ulcers.

Current surgical treatment options for venous valve repair of more severe cases of CVI include direct valve repair, bypass, and axillary vein valve transfer. Notably, there is no treatment option for abluminally restoring venous valve function via endovascular intervention.

Disclosed herein are systems and methods for endovascularly restoring venous valve function.

### SUMMARY

Disclosed herein is a system for restoring venous valve function. The system includes, in some embodiments, an implantable coil and a deployment device for the implantable coil. The implantable coil has a non-coiled configuration in a first state and a coiled configuration in a second state. The deployment device includes a handle, an outer sheath extending from the handle, a needle, and a mandrel. The handle includes a first actuator and a second actuator. The outer sheath includes a side opening proximate a distal end. The needle is operatively coupled to the first actuator. The mandrel is operatively coupled to the second actuator. The mandrel is configured to push the implantable coil through a needle tip of the needle, which allows the implantable coil to transition from the first state within the deployment device to the second state outside of the deployment device.

In some embodiments, the needle tip is configured to pierce a luminal surface of a vein from the side opening of the outer sheath. Piercing the luminal surface of the vein forms an opening in a wall of the vein.

In some embodiments, the implantable coil includes a locking bead at a proximal end thereof. The locking bead is configured to anchor the implantable coil in the opening of the wall of the vein, seal the opening in the wall of the vein, prevent migration of the implantable coil, or a combination thereof.

In some embodiments, the implantable coil is configured to wrap around an abluminal surface of the vein and constrict a valve area thereof. Constricting the valve area brings leaflets within the valve area closer together.

In some embodiments, the implantable coil is a shape-set nitinol implantable coil.

In some embodiments, the implantable coil includes one or more echogenicity-enhancing features for ultrasound visualization of the implantable coil.

In some embodiments, the implantable coil includes one or more radiopacity-enhancing feature for X-ray visualization of the implantable coil.

In some embodiments, the first actuator includes a first-actuator sliding element coupled to the needle. The sliding element is configured to distally slide in a first-actuator slot of the handle to advance the needle tip through the side opening of the outer sheath. The sliding element is also configured to proximally slide in the first-actuator slot of the handle to withdraw the needle tip into the outer sheath.

In some embodiments, the first-actuator sliding element include a deflection wheel coupled to the needle tip by a pull wire. The deflection wheel is configured to pull the pull wire by rotation of the deflection wheel for setting a needle-tip angle prior to advancing the needle tip through the side opening of the outer sheath.

In some embodiments, the needle is shape set with a curved shape in a distal-end portion of the needle for advancing the needle through the side opening of the outer sheath without setting a needle-tip angle prior to advancing the needle tip through the side opening of the outer sheath.

In some embodiments, the second actuator includes a second-actuator sliding element coupled to the mandrel. The second-actuator sliding element is configured to distally slide in a second-actuator slot of the handle to advance the mandrel and push the implantable coil through the needle tip.

Also disclosed herein is an implantable coil for restoring venous valve function. The implantable coil includes a coil body and a locking bead. The coil body has a non-coiled configuration in a first state of the implantable coil and a coiled configuration in a second state of the implantable coil. The second state of the implantable coil is configured to coil around an abluminal surface of a vein and constrict a valve area thereof to bring leaflets within the valve area closer together. The locking bead is at a proximal end of the coil body opposite a distal end thereof. The locking bead is configured to anchor the implantable coil to a luminal surface of the vein by way of an opening of a wall of the vein.

In some embodiments, the locking bead is configured to seal the opening in the wall of the vein and prevent migration of the implantable coil.

In some embodiments, the locking bead is formed of a bioresorbable material.

In some embodiments, the implantable coil is a shape-set nitinol implantable coil.

In some embodiments, the coil body is shaped into a circular cylinder with circular coils in the second state of the implantable coil.

In some embodiments, the coil body is shaped into an elliptic cylinder with elliptical coils in the second state of the implantable coil.

In some embodiments, the coil body is shaped into a cone with circular coils of increasing diameter from the proximal end to the distal end of the coil body in the second state of the implantable coil.

In some embodiments, the coil body is shaped into an hourglass with circular coils of decreasing diameter from both the proximal and distal ends of the coil body in the second state of the implantable coil.

In some embodiments, the coil body is shaped into a barrel with circular coils of increasing diameter from both the proximal and distal ends of the coil body in the second state of the implantable coil.

In some embodiments, the coil body is formed of a single wire.

In some embodiments, the coil body is formed of a number of wires braided together.

In some embodiments, the coil body is formed of a number of wires wound around a common core wire.

In some embodiments, the implantable coil includes one or more echogenicity-enhancing features for ultrasound visualization of the implantable coil.

In some embodiments, the implantable coil includes one or more radiopacity-enhancing feature for X-ray visualization of the implantable coil.

Also disclosed herein is a method for restoring venous valve function. The method includes, in some embodiments, system-obtaining step, a sheath-inserting step, a needle-advancing step, and an implantable coil-pushing step. The system-obtaining step includes obtaining a system for restoring venous valve function. The system includes, in some embodiments, an implantable coil and a deployment device for the implantable coil. The implantable coil has a non-coiled configuration in a first state and a coiled configuration in a second state. The deployment device includes a handle, an outer sheath extending from the handle, a needle, and a mandrel. The handle includes a first actuator and a second actuator. The outer sheath includes a side opening proximate a distal end. The needle is operatively coupled to the first actuator. The mandrel is operatively coupled to the second actuator. The mandrel is configured to push the implantable coil through a needle tip of the needle, which allows the implantable coil to transition from the first state within the deployment device to the second state outside of the deployment device. The sheath-inserting step includes inserting the outer sheath of the deployment device into a vein of a patient. The needle-advancing step includes actuating the first actuator to advance the needle tip through the side opening of the outer sheath and through a luminal surface of a wall of the vein. The implantable coil-pushing step includes actuating the second actuator to push the implantable coil through the needle tip. Upon being pushed through the needle tip, the implantable coil transitions from the first state to the second state around an abluminal surface of the wall of the vein.

In some embodiments, the needle-advancing step includes distally sliding a first-actuator sliding element coupled to the needle in a first-actuator slot of the handle.

In some embodiments, the method further includes a needle-withdrawing step. The needle-withdrawing step includes actuating the first actuator to withdraw the needle tip into the outer sheath. The needle-withdrawing step includes proximally sliding the first actuator in the first-actuator slot of the handle.

In some embodiments, the method further includes an angle-setting step. The angle-setting step includes actuating the first actuator to set a needle-tip angle of the needle tip prior to the needle-advancing step. The angle-setting step includes rotating a deflection wheel of the first-actuator sliding element to pull or release a pull wire coupled to the needle tip.

In some embodiments, the implantable coil-pushing step includes distally sliding a second-actuator sliding element in a second-actuator slot of the handle.

In some embodiments, the method further includes an implantable coil-anchoring step. The implantable coil-anchoring step includes anchoring the implantable coil in an opening in the wall of the vein by a locking bead at a proximal end of the implantable coil. The locking bead is configured to seal the opening in the wall of the vein and prevent migration of the implantable coil.

In some embodiments, the method further includes an ultrasound-imaging step. The ultrasound-imaging step includes imaging placement of the implantable coil around the abluminal surface of the wall of the vein by ultrasound. The implantable coil includes one or more echogenicity-enhancing features for ultrasound visualization of the implantable coil.

In some embodiments, the method further includes a fluoroscopy-imaging step. The fluoroscopy-imaging step includes imaging placement of the implantable coil around the abluminal surface of the wall of the vein by fluoroscopy. The implantable coil includes one or more radiopacity-enhancing features for X-ray visualization of the implantable coil.

In some embodiments, the method further includes a vein-imaging step and an implantable coil-sizing step. The vein-imaging step includes imaging the vein by ultrasound or fluoroscopy. The implantable coil-sizing step includes determining a proper size of the implantable coil for placing the implantable coil around the abluminal surface of the wall of the vein before the sheath-inserting step.

In some embodiments, the method further includes a sheath-withdrawing step. The sheath-withdrawing step includes withdrawing the outer sheath of the deployment device from the vein of the patient.

These and other features of the concepts provided herein will become more apparent to those of skill in the art in view of the accompanying drawings and following description, which describe particular embodiments of such concepts in greater detail.

### DRAWINGS

FIG. 1 illustrates a top view with partial cutaway of a first deployment device in accordance with some embodiments.

FIG. 2 illustrates a top view with partial cutaway of a second deployment device in accordance with some embodiments.

FIG. 3 illustrates a distal portion of a needle in accordance with some embodiments.

FIG. 4A illustrates a first implantable coil in accordance with some embodiments.

FIG. 4B illustrates a second implantable coil in accordance with some embodiments.

FIG. 4C illustrates a third implantable coil in accordance with some embodiments.

FIG. 4D illustrates a fourth implantable coil in accordance with some embodiments.

FIG. 4E illustrates a fifth implantable coil in accordance with some embodiments.

FIG. 4F illustrates a sixth implantable coil in accordance with some embodiments.

FIG. 5 illustrates a method of restoring valve function in accordance with some embodiments.

FIG. 6A illustrates an implanted implantable coil compressing an incompetent valve of a vein in accordance with some embodiments.

FIG. 6B illustrates a cross-sectional view of the implanted implantable coil compressing the incompetent valve of the vein in accordance with some embodiments.

FIG. 7A illustrates blood flow through a vein with a properly functioning valve in accordance with some embodiments.

FIG. 7B illustrates blood flow through a vein with an improperly functioning valve in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, current surgical treatment options for venous valve repair of more severe cases of CVI include direct valve repair, bypass, and axillary vein valve transfer. Notably, there is no treatment option for restoring venous valve function via endovascular intervention.

Disclosed herein are systems and methods for endovascularly restoring venous valve function. The systems include deployment devices and implantable coils, which are described, in turn, below. The methods include endovascularly restoring venous valve function with the systems or any one or more components thereof.

### Deployment Devices

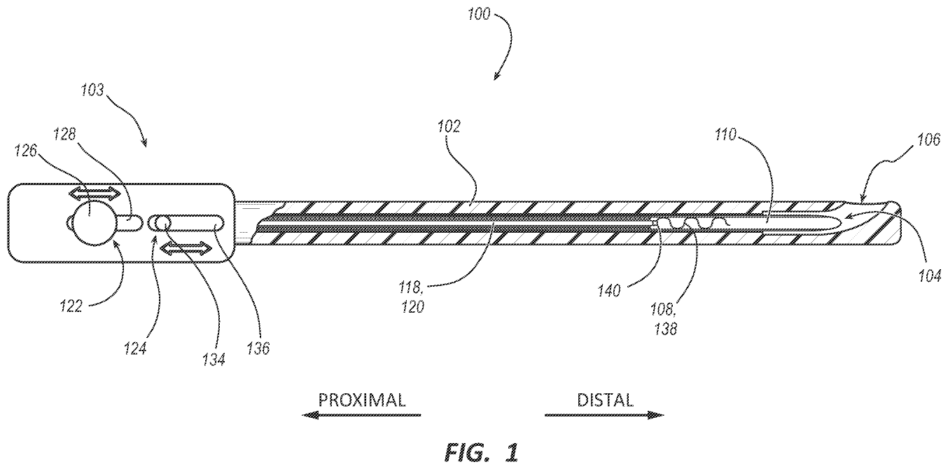

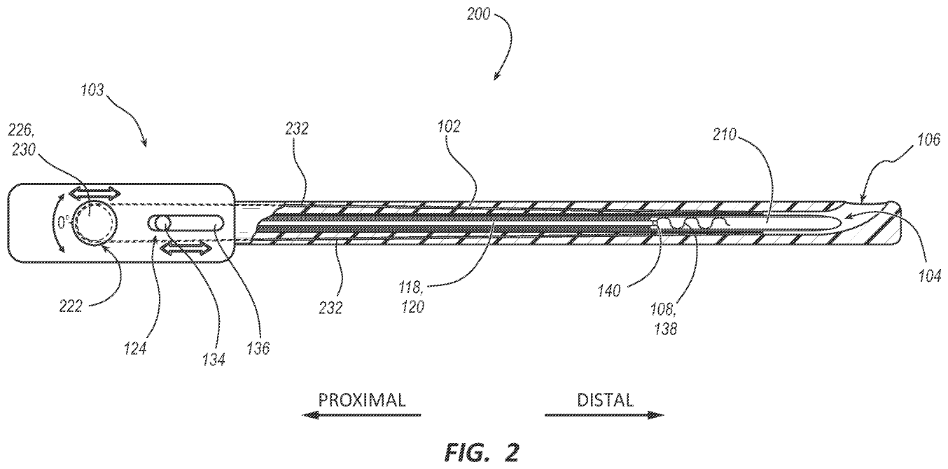

FIG. 1 illustrates a first deployment device 100 in accordance with some embodiments. FIG. 2 illustrates a second deployment device 200 in accordance with some embodiments.

A primary difference between the first deployment device 100 and the second deployment device 200 is that of a needle-tip angle-setting means for setting a needle-tip angle of the needle 110 or 210 set forth below. Indeed, the first deployment device 100 includes the shape-set needle 110 having a set needle-tip angle, whereas the second deployment device 200 includes the deflectable needle 210 and the deflection wheel 230 for setting the needle-tip angle. In view of the foregoing, the first deployment device 100 and the second deployment device 200 are primarily described using the same reference numbers for components unless components of the needle-tip angle-setting means are being described.

Each deployment device of the deployment devices 100 and 200 includes an outer sheath 102 extending from a handle 103 as shown in FIGS. 1 and 2.

The outer sheath 102 is configured to be disposed in a vein such as a leg vein (e.g., an upper or lower leg vein) or arm vein for endovascularly restoring valve function for one or more valves thereof. The outer sheath 102 includes a sheath lumen 104 and a side opening 106 proximate a closed distal end of the outer sheath 102. The sheath lumen 104 is configured to accommodate the needle 110 or 210 slidably disposed therein. The side opening 106 is configured to allow at least the needle tip 116 or 216 therethrough to pierce a luminal surface of a vein and form an opening in a wall of the vein for deploying an implantable coil 108 therearound. The outer sheath 102 is formed (e.g., extruded) of a compliant medical grade polymer with a tubular wall of sufficient thickness to provide structural support for advancing the needle 110 or 210 through the outer sheath 102 with the first actuator 122 or 222.

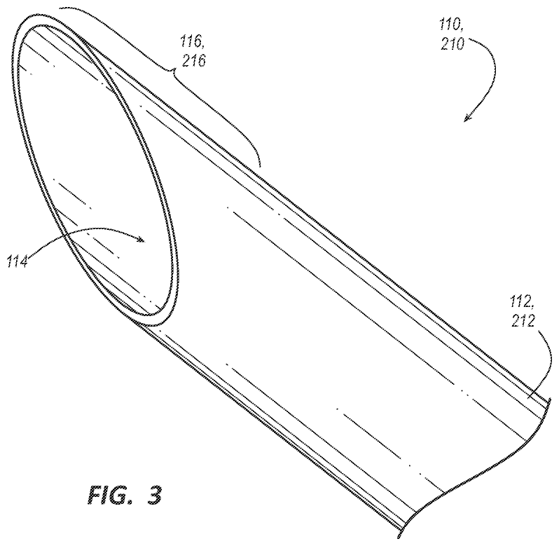

FIG. 3 illustrates a distal portion of a needle 110 or 210 in accordance with some embodiments.

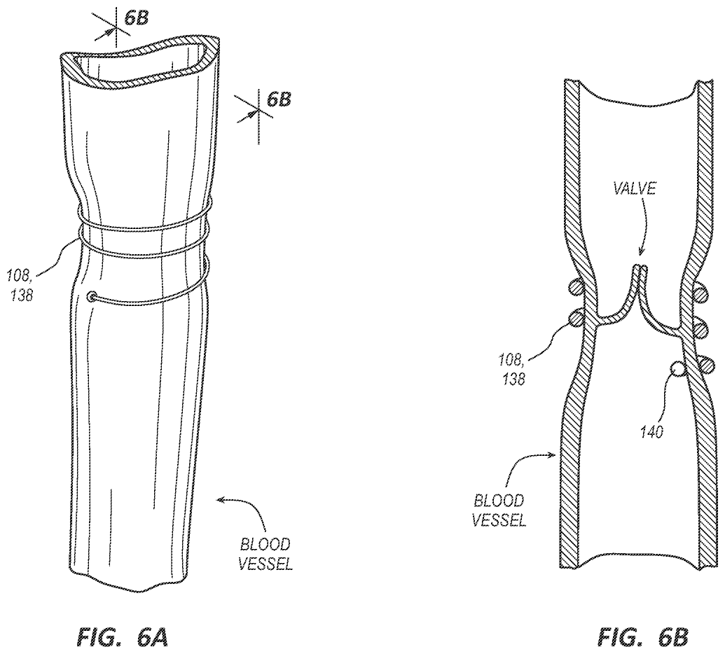

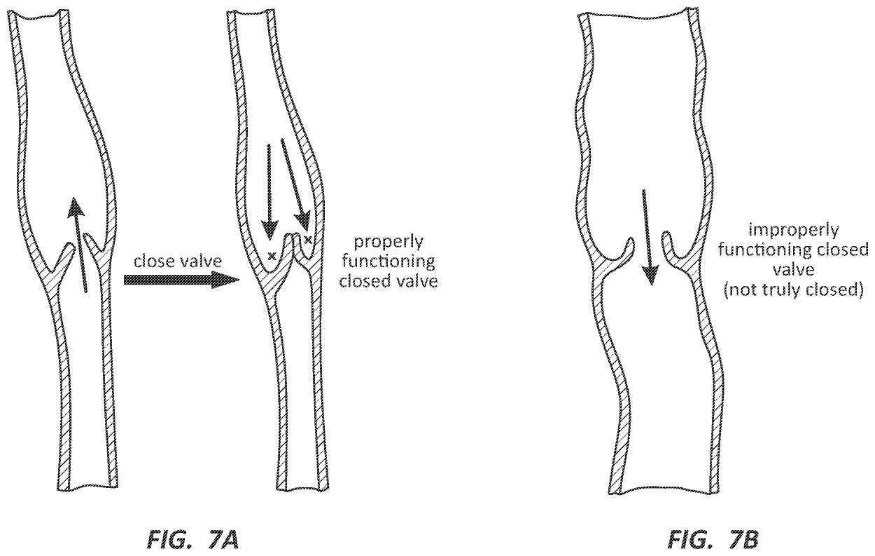

The needle 110 or 210 is configured to pierce a luminal surface of a vein (e.g., a leg vein, an arm vein, etc.) and form an opening in a wall of the vein for deploying the implantable coil 108 therearound. The needle 110 or 210 includes a needle shaft 112 or 212 having a needle lumen 114 and a needle tip 116 or 216 having a distal opening as shown in FIG. 3. The needle lumen 114 is configured to accommodate both a mandrel 118 and the implantable coil 108 slidably disposed therein, with the implantable coil 108 disposed in the needle lumen 114 distal of the distal end of the mandrel 118 in a ready-to-deploy state of the deployment device 100 or 200. The needle tip 116 or 216 can be precisely laser cut to pierce a luminal surface of a vein and form an opening in a wall of the vein. (For the opening in the wall of the vein, see the location of the locking bead 140 in FIG. 6B.) The distal opening is configured to allow at least the implantable coil 108 therethrough for deploying the implantable coil 108 around an abluminal surface of the vein. The needle 110 or 210 is formed of a medical grade metal (e.g., nitinol, stainless steel, etc.) with a tubular wall of sufficient thickness to support holding a shape-set curved shape of the needle 110 or deflecting the needle tip 216 of the needle 210 into the curved shape with the second actuator 124 for advancing the needle 110 or 210 through the side opening 106 of the outer sheath 102 with a particular needle-tip angle (i.e., the angle formed between a proximal portion of the needle shaft 112 or 212 and a distal portion of the needle shaft 112 or 212 including the needle tip 116 or 216).

The mandrel 118 is configured to push the implantable coil 108 through the needle tip 116 or 216, which allows the implantable coil 108 to transition from a first state within the needle 110 or 210 of the deployment device 100 or 200 to a second state outside of the deployment device 100 or 200 such as around an abluminal surface of a vein. The mandrel 118 includes a mandrel shaft 120 having a distal end configured to push the implantable coil 108 therewith. The mandrel 118 is formed of a medical grade metal (e.g., nitinol, stainless steel, etc.) or polymer with a diameter of sufficient thickness to support pushing the implantable coil 108 through the needle tip 116 or 216 without buckling.

The handle 103 is configured to be held by a clinician and operated thereby. For operating the deployment device 100 or 200, the handle 103 includes a first actuator 122 or 222 operatively coupled to the needle 110 or 210 and a second actuator 124 operatively coupled to the mandrel 118. The first actuator 122 and the needle 110 are described first followed by the first actuator 222 and the needle 210. The second actuator 124 and the mandrel 118 are described thereafter.

The first actuator 122 is configured to advance the needle tip 116 through the side opening 106 of the outer sheath 102, as well as withdraw the needle tip 116 into the outer sheath 102. The first actuator 122 includes a first-actuator sliding element 126 (e.g., a post including a larger-diameter post head) either directly coupled to the needle 110 or indirectly coupled to the needle 110. In an example of the former, the first-actuator sliding element 126 can include a through hole through which the proximal portion of the needle shaft 112 is inserted and directly coupled. In an example of the latter, the first-actuator sliding element 126 and the proximal portion of the needle shaft 112 can be indirectly coupled through an intervening extender of first actuator 122. The first-actuator sliding element 126 is configured to distally slide from a proximal position in a first-actuator slot 128 of the handle 103 in the ready-to-deploy state of the deployment device 100 to advance the needle tip 116 through the side opening 106 of the outer sheath 102. The first-actuator sliding element 126 is also configured to proximally slide from a distal position in the first-actuator slot 128 of the handle 103 in an operating state of the deployment device 100 to withdraw the needle tip 116 into the outer sheath 102.

The first actuator 122 and the needle 110 are configured to be used together. Due to the shape-set curved shape of the needle 110, when the first-actuator sliding element 126 is slid from the proximal position in the first-actuator slot 128 of the handle 103 to advance the needle tip 116 through the side opening 106 of the outer sheath 102, the needle 110 assumes the set needle-tip angle when the distal portion thereof is free from the sheath lumen 104. Thus, with the needle 110 there is not a need to set the needle-tip angle for the needle 110 prior to advancing the needle tip 166 through the side opening 106 of the outer sheath 102.

Like the first actuator 122, the first actuator 222 is configured to advance the needle tip 216 through the side opening 106 of the outer sheath 102, as well as withdraw the needle tip 216 into the outer sheath 102. Indeed, the first actuator 222 likewise includes a first-actuator sliding element 226 (e.g., a defection wheel-topped post) either directly coupled to the needle 210 or indirectly coupled to the needle 210. As set forth above, the first-actuator sliding element 226 is configured to distally slide from the proximal position in the first-actuator slot 128 (not shown in FIG. 2) of the handle 103 in the ready-to-deploy state of the deployment device 200 to advance the needle tip 216 through the side opening 106 of the outer sheath 102. The first-actuator sliding element 226 is also configured to proximally slide from the distal position in the first-actuator slot 128 of the handle 103 in an operating state of the deployment device 200 to withdraw the needle tip 216 into the outer sheath 102. However, the first-actuator sliding element 226 further includes a deflection wheel 230 coupled to the needle tip 216 by a pull wire 232. The deflection wheel 230 is configured to pull the pull wire 232 by rotation of the deflection wheel 230 for setting the needle-tip angle prior to advancing the needle tip 216 through the side opening 106 of the outer sheath 102.

The first actuator 222 and the needle 210 are configured to be used together. Due to a lack of a shape-set curved shape like that of the needle 110, before or while the first-actuator sliding element 226 is slid from the proximal position in the first-actuator slot 128 of the handle 103 to advance the needle tip 216 through the side opening 106 of the outer sheath 102, the deflection wheel 230 is rotated to set the set needle-tip angle for when the distal portion of the needle 210 is free from the sheath lumen 104. Thus, with the needle 210 there is a need to set the needle-tip angle for the needle 210 prior to advancing the needle tip 216 through the side opening 106 of the outer sheath 102.

The second actuator 124 is configured to advance the distal end of the mandrel 118 through the needle tip 116 or 216 of the needle 110 or 210, as well as withdraw the distal end of the mandrel 118 into the needle 110 or 210. In the ready-to-deploy state of the deployment device 100 or 200, the implantable coil 108 is disposed in the needle lumen 114 distal of the distal end of the mandrel 118 such that advancing the distal end of the mandrel 118 through the needle tip 116 or 216 deploys the implantable coil 108. The second actuator 124 includes a second-actuator sliding element 134 (e.g., a post) either directly coupled to the mandrel 118 or indirectly coupled to the mandrel 118. In an example of the former, the second-actuator sliding element 134 can include a through hole through which a proximal portion of the mandrel 118 is inserted and directly coupled. In an example of the latter, the second-actuator sliding element 134 and the proximal portion of the mandrel 118 can be indirectly coupled through an intervening extender of second actuator 124. The second-actuator sliding element 134 is configured to distally slide from a proximal position in a second-actuator slot 136 of the handle 103 in the ready-to-deploy state of the deployment device 100 or 200 to advance the distal end of the mandrel 118 and the implantable coil 108 through the needle tip 116 or 216 of the needle 110 or 210. The second-actuator sliding element 134 is also configured to proximally slide from a distal position in the second-actuator slot 136 of the handle 103 in an operating state of the deployment device 100 or 200 to withdraw the distal end of the mandrel 118 into the needle 110 or 210.

### Implantable Coils

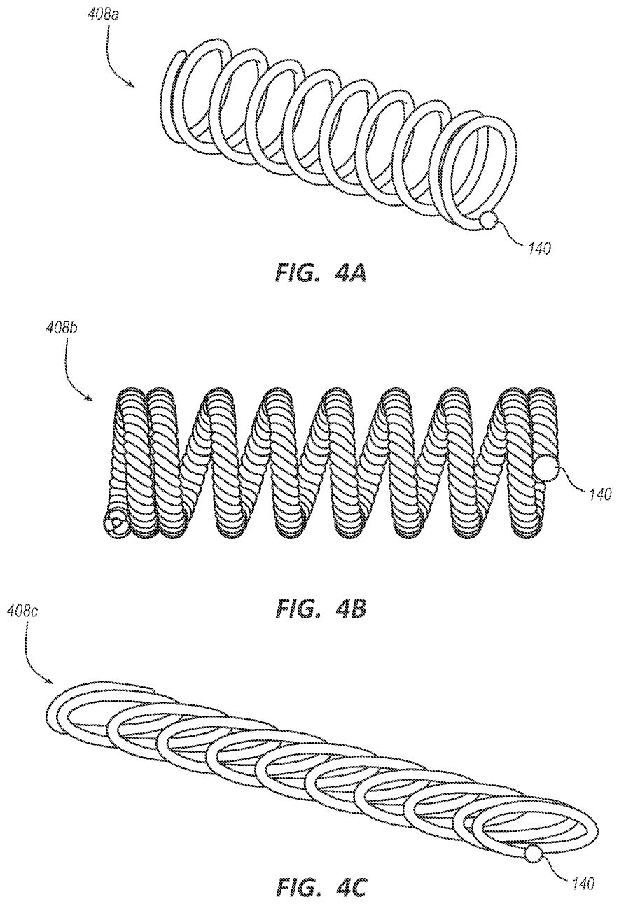

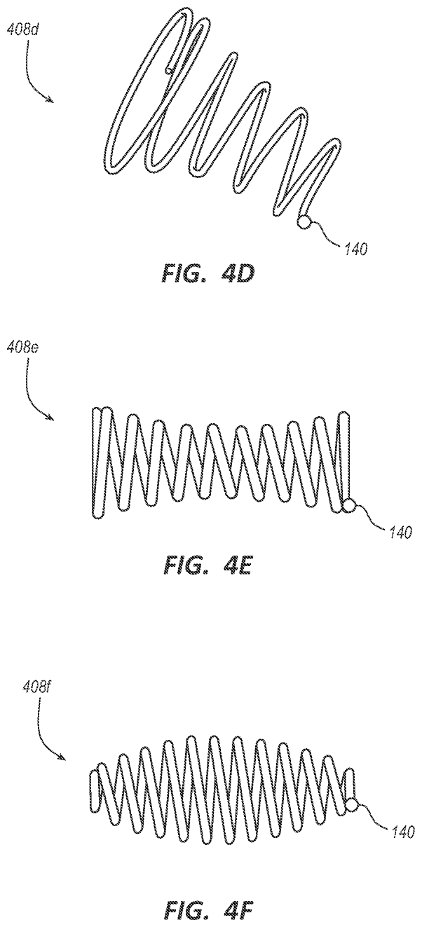

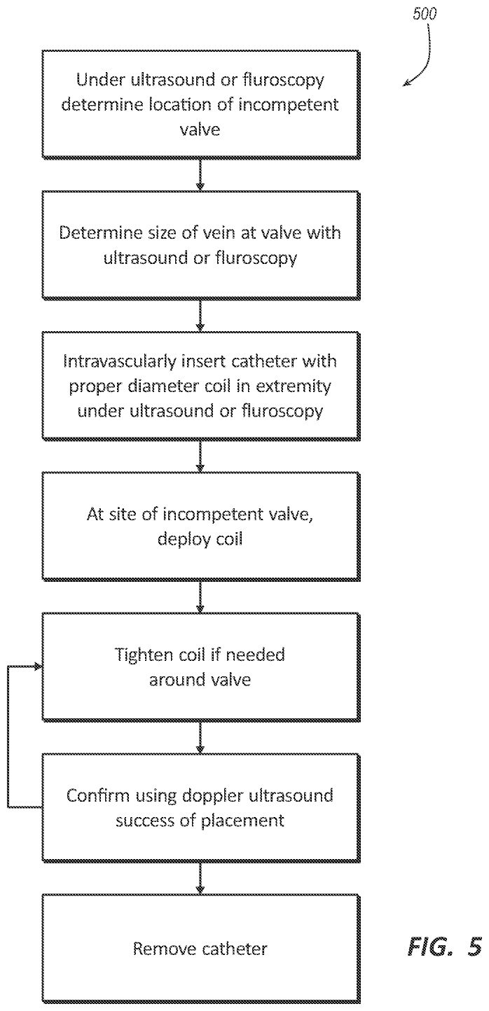

FIGS. 4A-4F illustrates various implantable coils 408a, 408b, 408c, 408d, 408e, and 408f in accordance with some embodiments. It should be understood the implantable coil 108 represents an implantable-coil genus, of which implantable coils 408a, 408b, 408c, 408d, 408e, and 408f are species. Any implantable coil of the implantable coils 408a, 408b, 408c, 408d, 408e, and 408f can be used with the deployment device 100 or 200.

The implantable coil 108 is configured to wrap around an abluminal surface of a vein and constrict a valve area thereof, which brings leaflets within the valve area closer together to restore proper valve functioning. The implantable coil 108 includes one or more wires coiled into a coiled body 138 having a locking bead 140 at a proximal end thereof. The coiled body 138 can be shaped into a circular cylinder with circular coils as shown by the implantable coils 408a and 408b respectively of FIGS. 4A and 4D, an elliptic cylinder with elliptical coils as shown by the implantable coil 408c of FIG. 4C, a cone with circular coils of increasing diameter from the proximal end or a distal end thereof as shown by the implantable coil 408d of FIG. 4D, an hourglass with circular coils of decreasing diameter from both the proximal and distal ends of the implantable coil 408e to a center thereof as shown in FIG. 4E, or a barrel with circular coils of increasing diameter from both the proximal and distal ends of the implantable coil 408f to a center thereof as shown in FIG. 4F. Like the implantable coil 408b of FIG. 4B, any of the foregoing implantable coils can include a number of wires braided together or wound around a common core wire and coiled into its respective coiled body. The locking bead 140 at the proximal end of the implantable coil 108 is configured to anchor the implantable coil 108 in an opening of the wall of the vein created by the needle tip 116 or 216, seal the opening in the wall of the vein, prevent migration of the implantable coil 108, or a combination thereof. As shown, the locking bead 140 includes a solid sphere; however, any solid mass at the proximal end of the implantable coil 108 is sufficient for the locking bead 140. The locking bead 140 can be of a same or different material as the coiled body 138. For example, the locking bead 140 can be formed of a bolus of bioresorbable material into which the proximal end of the one or more wires of the coiled body 138 is inserted.

The implantable coil 108 is formed of one or more medical grade metal wires (e.g., nitinol, stainless steel, etc.) coiled into the coiled body 138 of the implantable coil 108. One or more diameters of the one-or-more metal wires together with coil parameters such as a number of active coils in the implantable coil 108 and an outer diameter of the implantable coil 108 are selected for sufficiency in wrapping the implantable coil 108 around an abluminal surface of a vein and constricting a valve area thereof to restore proper valve functioning. The foregoing parameters are also selected for providing a non-coiled configuration in a first state of the implantable coil 108 and a coiled configuration in a second state of the implantable coil 108. The implantable coil 108 assumes the first, non-coiled state of the implantable coil 108 when disposed in the needle lumen 114 in the ready-to-deploy state of the deployment device 100 or 200. The implantable coil 108 assumes the second, coiled state of the implantable coil 108 when deployed outside of the deployment device 100 or 200 such as around an abluminal surface of a vein. The implantable coil 108 can include one or more echogenicity-enhancing features (e.g., a coating, surface scoring, surface patterning such as helical or sawtooth patterns, denting, or a combination thereof) for ultrasound visualization of the implantable coil 108. For example, the implantable coil 408b of FIG. 4B includes surface patterning (e.g., helical patterns) by virtue of the number of wires braided together or wound around the common core wire of the implantable coil 408b. Alternatively or additionally, the implantable coil 108 can include one or more radiopacity-enhancing features (e.g., radiopaque bands or arrows) for X-ray visualization of the implantable coil 108.

### Methods

Methods for endovascularly restoring venous valve function include methods of using the systems set forth above or any one or more components thereof such as the deployment device 100 or 200, the implantable coil 108 or any species thereof (e.g., implantable coils 408a-408f, or some combination thereof.

FIG. 5 illustrates a method 500 of restoring valve function in accordance with some embodiments.

As shown, the method 500 includes at least a sheath-inserting step, an implantable coil-pushing step, and a sheath-withdrawing step. However, the method 500 is not limited thereto. Indeed, the method 500 further includes at least a system-obtaining step and a needle-advancing step after the sheath-inserting step, as well as any of a number of optional steps as set forth below.

The system-obtaining step includes obtaining the system for restoring venous valve function. As set forth above, the system includes the implantable coil 108 and the deployment device 100 or 200 for the implantable coil 108. The implantable coil 108 has the non-coiled configuration in the first state and the coiled configuration in the second state. The deployment device 100 or 200 includes the handle 103, the outer sheath 102 extending from the handle 103, the needle 110 or 210, and the mandrel 118. The handle 103 includes the first actuator 122 or 222 and the second actuator 124. The outer sheath 102 includes the side opening 106 proximate the closed distal end. The needle 110 or 210 is operatively coupled to the first actuator 122 or 222. The mandrel 118 is operatively coupled to the second actuator 124. The mandrel 118 is configured to push the implantable coil 108 through the needle tip of the needle 110 or 210, which allows the implantable coil 108 to transition from the first state within the deployment device 100 or 200 to the second state outside of the deployment device 100 or 200.

The method 500 further can further include a vein-imaging step and an implantable coil-sizing step as show in FIG. 5. The vein-imaging step includes imaging a vein of a patient by ultrasound or fluoroscopy to determine a location of a valve in need of valve-function restoration, as well as to determine a size of the vein including the valve. The implantable coil-sizing step includes determining a proper size of the implantable coil 108 for placing the implantable coil 108 around the abluminal surface of a wall of the vein before the sheath-inserting step.

The sheath-inserting step includes inserting the outer sheath 102 of the deployment device 100 or 200 into the vein of the patient after a percutaneous puncture and any needed insertion-site dilation. The sheath-inserting step can include advancing the outer sheath 102 to the valve in need of the valve-function restoration, optionally with ultrasonic or fluoroscopic imaging.

The needle-advancing step includes actuating the first actuator 122 or 222 to advance the needle tip 116 or 216 through the side opening 106 of the outer sheath 102 and through a luminal surface of the wall of the vein. Actuating the first actuator 122 or 222 of the needle-advancing step includes distally sliding the first-actuator sliding element 126 or 226 coupled to the needle 110 or 210 in the first-actuator slot 128 of the handle 103. If using the deployment device 200, however, the method 500 can further include an angle-setting step before the needle-advancing step. The angle-setting step includes actuating the first actuator 222 to set a needle-tip angle of the needle tip 216 prior to the needle-advancing step. Actuating the first actuator 222 of the angle-setting step includes rotating the deflection wheel 230 of the first-actuator sliding element 226 to pull or release the pull wire 232 coupled to the needle tip 216.

FIGS. 6A and 6B illustrate an implanted implantable coil 108 compressing the valve in need of the valve-function restoration in accordance with some embodiments.

The implantable coil-pushing step includes actuating the second actuator 124 to push the implantable coil 108 through the needle tip 116 or 216. Actuating the second actuator 124 of the implantable coil-pushing step includes distally sliding the second-actuator sliding element 134 in the second-actuator slot 136 of the handle 103. Upon being pushed through the needle tip 116 or 216, the implantable coil 108 transitions from the first state to the second state around an abluminal surface of the wall of the vein as shown in FIGS. 6A and 6B.

The method 500 further includes an implantable coil-anchoring step. The implantable coil-anchoring step includes anchoring the implantable coil 108 in an opening in the wall of the vein created by the needle tip 116 or 216 by the locking bead 140 at the proximal end of the implantable coil 108. As set forth above, the locking bead 140 is configured to seal the opening in the wall of the vein and prevent migration of the implantable coil 108.

The method 500 can further include an implantable coil-tightening step as shown in FIG. 5. The implantable coil-tightening step includes tightening the implantable coil 108 (e.g., by a ratchet-type mechanism) if needed to further constrict the valve and bring valve leaflets thereof closer together to restore valve function.

Notwithstanding any other ultrasound-imaging steps set forth above, the method 500 can further include an ultrasound-imaging step that includes imaging placement of the implantable coil 108 around the abluminal surface of the wall of the vein by ultrasound. As set forth above, the implantable coil 108 includes one or more echogenicity-enhancing features for ultrasound visualization of the implantable coil 108.

Notwithstanding any other fluoroscopy-imaging steps set forth above, the method 500 can further include a fluoroscopy-imaging step that includes imaging placement of the implantable coil 108 around the abluminal surface of the wall of the vein by fluoroscopy. As set forth above, the implantable coil 108 includes one or more radiopacity-enhancing features for X-ray visualization of the implantable coil 108.

The method 500 further includes a needle-withdrawing step. The needle-withdrawing step includes actuating the first actuator 122 or 222 to withdraw the needle tip 116 or 216 into the outer sheath 102. Actuating the first actuator 122 or 222 of the needle-withdrawing step includes proximally sliding the first-actuator sliding element 126 or 226 in the first-actuator slot 128 of the handle 103.

The sheath-withdrawing step includes withdrawing the outer sheath 102 of the deployment device 100 or 200 from the vein of the patient.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for restoring venous valve function, comprising:
an implantable coil having a non-coiled configuration in a first state and a coiled configuration in a second state, the implantable coil having a coil body extending between a distal end and a proximal end and defining a first diameter therebetween, and a locking bead coupled to the proximal end of the coil body and aligned axially therewith, the locking bead extending radially from a central axis of the coil body to define a second diameter, larger than the first diameter; and
a deployment device, comprising;
a handle including a first actuator and a second actuator;
an outer sheath extending from the handle, the outer sheath including a side opening proximate a distal end;
a needle operatively coupled to the first actuator; and
a mandrel operatively coupled to the second actuator, the mandrel configured to push the implantable coil through a needle tip of the needle, thereby allowing the implantable coil to transition from the first state within the deployment device to the second state outside of the deployment device.

2. The system of claim 1, wherein the needle tip is configured to pierce a luminal surface of a vein from the side opening of the outer sheath and form an opening in a wall of the vein.

3. The system of claim 2, wherein the locking bead is configured to anchor the implantable coil in the opening of the wall of the vein, seal the opening in the wall of the vein, prevent migration of the implantable coil, or a combination thereof.

4. The system of claim 3, wherein the implantable coil is configured to wrap around an abluminal surface of the vein and constrict a valve area thereof, thereby bringing leaflets within the valve area closer together.

5. The system of claim 4, wherein the implantable coil is a shape-set nitinol implantable coil.

6. The system of claim 5, wherein the implantable coil includes one or more echogenicity-enhancing features for ultrasound visualization of the implantable coil.

7. The system of claim 6, wherein the implantable coil includes one or more radiopacity-enhancing feature for X-ray visualization of the implantable coil.

8. The system of claim 7, wherein the first actuator includes a first-actuator sliding element coupled to the needle, the sliding element configured to distally slide in a first-actuator slot of the handle to advance the needle tip through the side opening of the outer sheath and proximally slide in the first-actuator slot of the handle to withdraw the needle tip into the outer sheath.

9. The system of claim 8, wherein the first-actuator sliding element includes a deflection wheel coupled to the needle tip by a pull wire, the deflection wheel configured to pull the pull wire by rotation of the deflection wheel for setting of a needle-tip angle prior to advancing the needle tip through the side opening of the outer sheath.

10. The system of claim 8, wherein the needle is a shape set with a curved shape in a distal-end portion of the needle for advancing the needle through the side opening of the outer sheath without setting a needle-tip angle prior to advancing the needle tip through the side opening of the outer sheath.

11. The system of claim 10, wherein the second actuator includes a second-actuator sliding element coupled to the mandrel, the second-actuator sliding element configured to distally slide in a second-actuator slot of the handle to advance the mandrel and push the implantable coil through the needle tip.

12. (canceled)

13. An implantable coil for restoring venous valve function, comprising:
a coil body defining a first diameter and having a non-coiled configuration in a first state of the implantable coil and a coiled configuration in a second state of the implantable coil for coiling around an abluminal surface of a vein and constricting a valve area thereof to bring leaflets within the valve area closer together; and
a locking bead at a proximal end of the coil body opposite a distal end thereof, the locking bead aligned axially with a central axis of the coil body and defining a second diameter larger than the first diameter, the locking bead configured to anchor the implantable coil to a luminal surface of the vein by way of an opening of a wall of the vein.

14. The implantable coil of claim 13, wherein the locking bead is configured to seal the opening in the wall of the vein and prevent migration of the implantable coil.

15. The implantable coil of claim 14, wherein the locking bead is formed of a bioresorbable material.

16. The implantable coil of claim 15, wherein the implantable coil is a shape-set nitinol implantable coil.

17. The implantable coil of claim 16, wherein the coil body is shaped into a circular cylinder with circular coils in the second state of the implantable coil.

18. The implantable coil of claim 16, wherein the coil body is shaped into an elliptic cylinder with elliptical coils in the second state of the implantable coil.

19. The implantable coil of claim 16, wherein the coil body is shaped into a cone with circular coils of increasing diameter from the proximal end to the distal end of the coil body in the second state of the implantable coil.

20. The implantable coil of claim 16, wherein the coil body is shaped into an hourglass with circular coils of decreasing diameter from both the proximal and distal ends of the coil body in the second state of the implantable coil.

21. The implantable coil of claim 16, wherein the coil body is shaped into a barrel with circular coils of increasing diameter from both the proximal and distal ends of the coil body in the second state of the implantable coil.

22. The implantable coil of claim 21, wherein the coil body is formed of a single wire.

23. The implantable coil of claim 21, wherein the coil body is formed of a number of wires braided together.

24. The implantable coil of claim 21, wherein the coil body is formed of a number of wires wound around a common core wire.

25. The implantable coil of claim 24, wherein the implantable coil includes one or more echogenicity-enhancing features for ultrasound visualization of the implantable coil.

26. The implantable coil of claim 25, wherein the implantable coil includes one or more radiopacity-enhancing feature for X-ray visualization of the implantable coil.

27-36. (canceled)
